(19) United States
(12) Patent Application Publication
(10) Pub. No.: US 2006/0008863 A1
(43) Pub. Date: Jan. 12, 2006

(54) Electrochemical sensing using an enzyme electrode

(75) Inventors: Brian Philip Allen, Toppesfield (GB); Xiao-Feng Zhou, Chelmsford (GB); Richard Gilbert, Chelmsford (GB)

Correspondence Address: SEED INTELLECTUAL PROPERTY LAW GROUP PLLC, 701 FIFTH AVE, SUITE 6300, SEATTLE, WA, 98104-7092, US

(73) Assignee: E2V Technologies (UL) Limited, Chelmsford (GB)

(21) Appl. No.: 10/519,518

(22) PCT Filed: Jun. 27, 2003
(86) PCT No.: PCT/GB03/02756

§ 371 Date: Jul. 18, 2005

(30) Foreign Application Priority Data
Jun. 28, 2002 (GB) 0214993.8
Apr. 30, 2003 (GB) 0309891.0

(51) Int. Cl. C12Q 1/26; G01N 33/50

(52) U.S. Cl. 435/25; 205/777.5

(57) ABSTRACT

Electrodes for use in electrochemical assays to determine whether a candidate drug is metabolised by an oxidative drug-metabolising enzyme (DME) are described. In a first type of electrode the DME is immobilised at the surface of the electrode. In a second type of electrode, a surface of the electrode is modified by the covalent or non covalent addition of chemical groups to allow efficient transfer of electrons from the electrode to the DME in solution. Use of the electrodes in electrochemical assays are described, as well as electrochemical reaction chambers comprising the electrodes.

This invention relates to electrodes for use in electrochemical assays, for example to determine whether a candidate drug is metabolised by an oxidative drug-metabolising enzyme (DME), and to use of the electrodes in such assays.

### SUMMARY

A key area of interest in the pharmaceutical industry is the prediction of how drugs are metabolised in the body. One of the main drug metabolism processes, phase I oxidation, is mediated by either the cytochrome P450 [CYP] or flavin monooxygenase (FMO) families of enzymes. The reactions catalysed by these enzymes can be driven electrochemically, and the reaction progress may be monitored using simple electrode systems. This makes the CYPs, and the functionally-related FMOs ideal candidates for electrochemical sensing.

### BACKGROUND

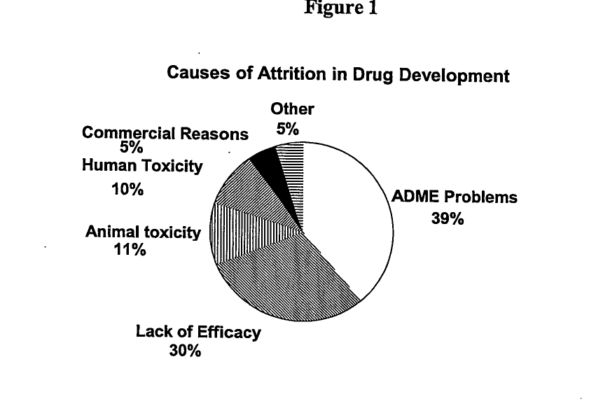

The single most important decision in the drug design process is made when selecting which of the lead compounds identified during the research programme are to be passed into the development pipeline. About 90% of development candidates fail to become marketed drugs, for a variety of reasons (see FIG. 1). The cost of a development programme is extremely high (typically £50M per molecule), and so the selection of development candidates has a high financial penalty if made incorrectly. For this reason, there is intense interest within the pharmaceutical industry for effective means to ‘fail drugs early’, identifying compounds that are unlikely to make it to market before vast expenditure is incurred.

The greatest single reason for a drug candidate to fail during development is for it to show unacceptable characteristics when introduced into live animals or humans. The collective term for these characteristics is ADME/Tox (absorption, distribution, metabolism, excretion and toxicity), covering how well a molecule enters the body, is distributed among the various tissues, is biochemically processed, and then eliminated in the bile or urine, as well as any unexpected toxicological effects that may be uncovered during the development and clinical trials programmes. ADME/Tox prediction is a current area of intense research, and will be an expanding market over the next 3-5 years.

Electrochemical sensing has a role to play in several areas within the ADME/Tox area, since many of the drug metabolism processes involve changes in redox potential. It therefore provides a means to quantity drug molecules, and their metabolic effects, in the context of a whole tissue or body fluid sample. In particular, it provides a sensitive and cost-effective means to follow the metabolic processing of drugs either at the single-enzyme, or whole organ level.

The drug-metabolising enzymes [DMEs] are a diverse group of proteins that are responsible for detoxifying a vast array of xenobiotic compounds (‘foreign molecules’) including drugs, pesticides and environmental pollutants. Most have an extremely broad substrate specificity: some individual members of the cytochrome P450 [CYP] and flavin monooxygenase [FMO] families are known to metabolise more than 50 structurally diverse compounds. Understanding the structure-activity relationships for the DMEs and their substrates is an important area of research that impacts on pharmacology, toxicology, and basic enzymology. In particular, the ability to predict whether a molecule is likely to be processed by CYPs in the body is of crucial importance in selecting candidate drug molecules for pharmaceutical development.

Conceptually, the DMEs are divided into two groups. Oxidative drug-metabolising enzymes, which include CYPs and FMOs, catalyse the introduction of an oxygen atom into substrate molecules, generally resulting in hydroxylation or demethylation. These enzymes are redox-driven, and the reactions they catalyse may readily be followed electrochemically. The conjugative enzyme families, which include the UDP-glycosyltransferases, glutathione transferases, sulphotransferases, and N-acetyltransferases, catalyse the coupling of endogenous small molecules to xenobiotics. This usually results in the formation of soluble compounds that are more readily excreted. The conjugative enzymes are not redox-driven, and are therefore not particularly suitable for electrochemical sensing. Other, as yet unidentified DMEs may also be found as a result of the human genome project. For example, the recently-discovered CYP3A34 has now been shown to be expressed in certain body tissues, although its precise function is currently unclear. The discussion here is therefore not limited to the enzymes explicitly mentioned in the text.

The CYP and FMO oxidative drug-metabolising enzymes are of particular interest as the biosensor components of an electrochemical device since the electrons they require to drive the reactions can be supplied by direct charge transfer from electrodes in a bioreactor chamber. Indeed, in such a device there is no requirement for additional biological or chemical components such as the cofactors and ancillary oxidoreductase enzymes that are necessary for driving the reactions in a conventional in vitro assay.

Proposed Biosensor Components

Cytochrome P450

CYP enzymes catalyse the initial step in the biotransformation of xenobiotic compounds, including most drugs (a process referred to as first-pass or Phase I metabolism). These enzymes are members of a large family of mixed-function oxidases which typically introduce an oxygen atom into substrate molecules, hence facilitating further metabolic processing to break the compound down. More than fifty CYP isozymes are known to exist in humans and they have been classified into 17 families and 39 subfamilies. In the standard nomenclature, the family is designated by a number, a letter designation for the subfamily, and a second number that identifies the individual member of that subfamily.

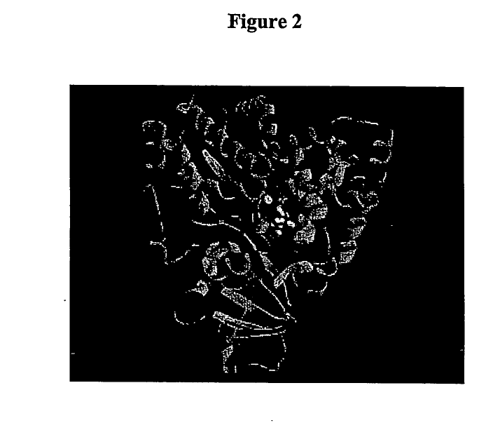

The 3D molecular structure of CYP2C9 showing the haem group, active-site iron atom and a bound substrate is shown in FIG. 2.

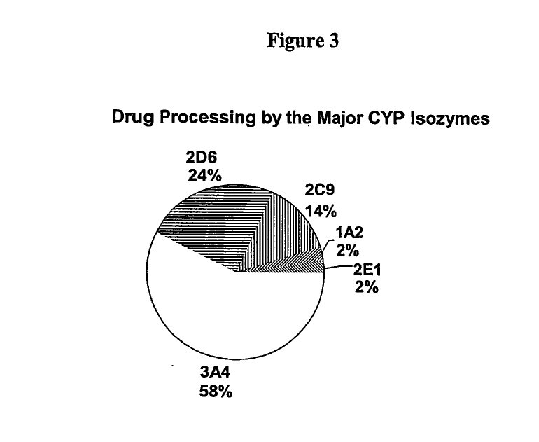

In humans and animals, the bulk of drug metabolism is carried out by just a few members of the CYP1, 2, and 3 families and occurs primarily in the liver, which contains the highest concentration of CYP in the body. FIG. 3 shows the percentage of drugs metabolised by the different CYP families. CYP3A4, 2D6, 1A2 and 2C9 are responsible for most of the drug metabolism by CYPs in humans, so are considered the most interesting from a drug screening point of view.

The oxidation of organic molecules by CYPs is quite complex, but the overall reaction can be simplified to the following equation:
RH+O₂+NADPH+H⁺→ROH+H₂O+NADP⁺

An electron from the cofactor NADPH (a common electron-transfer molecule) is transferred to a haem group within the CYP, where the activation of molecular oxygen occurs. Substrates (represented as R in the above equation) react with one of the oxygen atoms whilst the other is reduced to water, requiring a second electron. Several studies have shown that the electrons necessary to drive this reaction can be supplied electrochemically, with direct charge transfer coming from electrodes in an anaerobic bioreactor. In this case, there is no requirement for the NADPH cofactor. CYP enzymes are therefore ideal candidates for incorporation into an electrochemical sensor for predicting drug metabolism.

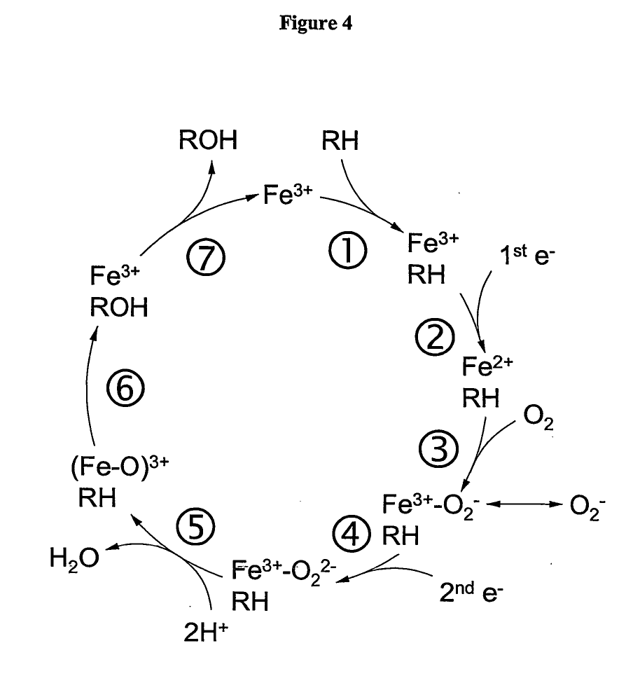

The generally accepted Cyp catalytic cycle is shown in FIG. 4. The reaction begins when the substrate binds to the active site (1). If the reaction is to proceed further, the substrate must displace a water molecule that is normally coordinated to the haem iron atom in unbound Cyp. This is accompanied by a change in the spin of the Fe³⁺ ion from a low spin (1/2) state in which the five 3d electrons are maximally paired, to a high spin (5/2) state in which the electrons are maximally unpaired. This in turn causes a change in the redox potential of the iron of approximately 100 mV, which is sufficient to make the reduction of the iron by the redox-partner of the Cyp (usually NADPH or NADH) thermodynamically favourable (2). The reduction step is followed by the binding of an O₂ molecule to a separate site adjacent to the Fe³⁺ ion (3). This state is not stable, and is easily autooxidised releasing O₂⁻. If, however, the transfer of a second electron occurs (4), the catalytic reaction continues. The O₂²⁻ reacts with protons from the surrounding solvent to form H₂O (which is released), leaving an activated oxygen atom (5). This may then react with the substrate molecule (6) resulting in a hydroxylated form of the substrate (7) which is then released from the active site.

The electrons which drive this reaction cycle are normally supplied in vivo by redox partners with the aid of appropriate oxidoreductase enzymes. In the case of the DMEs, the redox partner is usually nicotinamide adenosine dinucleotide phosphate (NADPH), which switches between oxidised (NADP+) and reduced states. Current in vitro DME assays require a reasonably complex reaction mixture which is able to regenerate the redox partners in the appropriate oxidation state.

Flavin Monooxygenases

Flavin monooxygenases, like the CYP enzymes, catalye the oxidation of organic compounds using molecular oxygen and NADPH as the source of electrons for the reduction of one of the oxygen atoms. However, they are mechanistically distinct from the CYPs in that they react with oxygen and NADPH in the absence of substrate to adopt an activated state within the cell, and an interaction with a nucleophilic group such as an amine, thiol, or phosphate is all that is required for completion of the catalytic cycle.

The capacity to remain stable whilst poised in an activated state is a possible explanation for the extremely broad substrate specificity of the FMO isozymes. It has been proposed that essentially all of the energy required for catalysis is captured in the oxygen-activated intermediate, and that alignment or distortion of the substrate molecules is not required, unlike most other enzymes. It follows that the active sites of FMOs are much less sterically defined than for other enzymes, allowing a wide variety of molecules to act as substrates. FMO3 is the most abundant form in human liver and is believed to be the dominant member of this enzyme family in terms of overall drug metabolism.

As for CYPs, it is possible to drive the FMO-mediated reactions by supplying electrons electrochemically, and therefore these would also be ideal candidates for incorporation into an electrochemical sensor device for predicting drug metabolism.

Despite the suitability of the oxidative DMEs for incorporation into electrochemical sensors for predicting drug metabolism, they have not yet been fully exploited electrochemically. In order to study the kinetics of oxidative DME-mediated reactions electrochemically, the rate-limiting step must be the oxidative DME-catalysed reaction, and not the transfer of electrons onto the enzyme. Due to slow mass-transfer at the electrode surface caused by the relatively low diffusion rate of the large oxidative DME molecules, it has not been possible to obtain accurate kinetic data.

One of the most important aspects of driving an enzyme-catalysed reaction electrochemically is the efficient transfer of electrons from the electrode(s) to the catalytic site within the enzyme. One way to maximise this transfer is to immobilise the enzyme at the surface of the electrode.

According to a first aspect of the invention there is provided an electrode comprising an oxidative drug-metabolising enzyme (DME) immobilised at the surface of the electrode to allow efficient transfer of electrons from the electrode to a catalytic site within the oxidative DME.

Efficient transfer of electrons from the electrode to the catalytic site within the DME occurs if the rate of transfer is at least as fast as the rate of consumption of electrons by the DME when metabolising a candidate drug. If metabolism of the candidate drug is limited by the transfer of electrons, accurate measurement of the rate of turnover of the candidate drug by the DME is not possible since electron transfer to the DMR then becomes the rate-limiting step.

Typically, a DME molecule will turnover approximately 10-100 substrate molecules per second. According to the Cyp catalytic mechanism two electrons are consumed for each molecule of substrate that is turned over. Thus, the electrode should be capable of transferring electrons to the DME at a rate of at least 20 electrons per second, more preferably at least 40 electrons per second, most preferably at least 200 electrons per second.

The electrode may be any suitable electrically conductive material, preferably graphite or metal, most preferably gold.

The DME may be covalently or non-covalently immobilised to the surface of the electrode.

The DME may be immobilised to the electrode by means of a linker. The linker may be covalently or non-covalently immobilised to the electrode, and covalently or non-covalently attached to the DME.

The linker should allow efficient transfer of electrons from the electrode to the catalytic site within the DME. Preferably the linker comprises a delocalised electron system.

Preferably the linker comprises one or more electrode binding groups to immobilise the DME to the electrode. The binding group should allow formation of a stable bond with the electrode at the operating voltage of the electrode. Preferred metal binding groups for binding to metal electrodes include amides, amines, carboxylic acids, and heterocyclic groups such as thiophenes, or nitrogen containing heterocyclic groups such as pyridines, purines, or pyrimidines. For a gold electrode, suitable functional groups include (but are not limited to) thiols, thioethers, thiophenes, pyridine, nitrogen-containing heterocycles, carboxylic acids and most negatively-charged moieties.

The physico-chemical properties of the linker must match the properties of the DME immobilised to the electrode. In particular the Gibbs free energy of interaction of the linker with the DMB must be favourable. Both the change in enthalpy and entropy on binding are important.

Preferred functional groups which contribute to the enthalpy of binding include hydrogen bond donors and acceptors, such as hydroxyls, amines, amides, carboxylic acids, aromatic systems, heterocycles, enols, ethers, ketones, aldehydes, thiols, thioethers, plus halo-, nitro-, phospho- and sulphate groups, or thiol equivalents of these groups. Preferably the linker comprises at least two or three of these groups.

Preferred functional groups which contribute to the entropy of binding include amines, amides, carboxylic acids, aromatic systems, cyclic groups, particularly heterocycles, enols, ethers, ketones, aldehydes, thiols, thioethers, plus halo-, nitro-, phospho- and sulphate groups.

Many classes of organic molecule provide suitable linkers. These include (but are not limited to) metallocenes, flavins, quinones, and NADH.

Preferred linkers comprise metallocenes, in particular ferrocenes. Cobalt metallocenes and vanadium metallocenes are also preferred.

Metallocenes have an unusual structure, in that a transition metal ion is sandwiched between two aromatic rings, such as the negatively charged cyclopentadienyl ion:

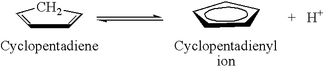

Two cyclopentadienyl rings can coordinate to an Fe²⁺ ion to form ferrocene, which may exist in either an oxidised or reduced state, thereby mirroring the characteristics of iron in the active site of the DUE haem groups:

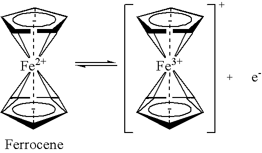

The ferrocenes in particular have appropriate redox potentials to be efficient transferors of charge for DMEs. They may carry substituent groups which can be used to optimise their binding characteristics to the enzymes and in addition they may be functionalised with appropriate chemical groups to allow them to bind tightly to the surface of the electrode.

There are several positions on the ferrocene skeleton which may be functionalised by the addition of chemical groups in order to modulate the molecule's redox potential and other physico-chemical characteristics such as shape, size, hydrophobicity, charge, and so on. These positions are indicated by the labels R₁ to R₁₀ in the Markush structure below.

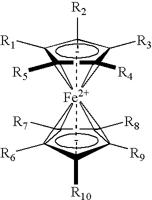

In ferrocene itself, all ten substituent positions are occupied by single hydrogen atoms. The substituent positions need not be independent. For example R₁ and R₂ might be joined together via a ring structure. The R positions are therefore simply indicators of where it is possible to vary the chemistry around the ferrocene core.

At least one of the potential R groups carries a suitable functional group for binding to the electrode. For example, it is well known that metallic gold has a particularly strong affinity for sulphur-containing groups such as thiols. If one of the R groups carries a thiol, it should therefore confer a strong gold-binding ability to the molecule. Provided this binding group is also able to support the ready transfer of electrons from the site of metal binding to the co-ordinated transition metal ion at the heart of the sandwich structure (e.g. by containing a delocalised electron system), then the linker should still have the ability to supply electrons to the DME. There are many alternatives to a thiol group for binding to a gold electrode, and many alternatives to gold as the electrode material.

Use of thiol containing groups for the linker is preferred where the DME is a flavin monooxygenase, or an oxidative DME other than a cytochrome P450.

Other examples of suitable ways to immobilise a DME to the surface of an electrode in accordance with the invention are described below.

Protein-Electrode Interactions

Immobilised Proteins

One of the most important aspects of driving an enzyme-catalysed reaction electrochemically is the efficient transfer of electrons from the electrode(s) to the catalytic site within the enzyme. One way to maximise this transfer is to immobilise the enzyme at the surface of the electrode. Although a system involving a solubilised enzyme could be designed, early success is most likely by using surface-immobilised enzymes.

Covalently-Modified Electrodes

The surface of an electrode of, for example, metal (typically, though not exclusively gold) or graphite, can be modified by the covalent addition of chemical groups to make it more amenable for the transfer of electrons to proteins. One technique involves the use of organothiloate compounds (containing an SH group) in conjunction with a gold electrode. The thiol group forms a strong bond to the metal surface, with the rest of the molecule providing suitable functional groups for interacting with the protein.

Microporous Electrolyte Membranes

These are mechanically and chemically stable polymer gels with high ionic conductivity, coating the surface of an electrode in the form of a thin layer. The polymers comprising the gel should be chosen to provide a suitable environment for trapping the proteins within their matrix, such as a high proportion (typically at least 50%) of carboxylic acid groups (for proteins with many positively-charged surface residues), amine groups (for proteins with many negative charges at the surface), or aliphatic groups (for proteins with largely hydrophobic surfaces, such as the CYPs).

The membrane should be mechanically and chemically stable enough that it remains physically intact and chemically unmodified at least for the duration of the experiment in which the electrode is used.

The ionic conductivity of the polymer gel should be high enough that electrons can be transferred from the electrode to the DME at a rate which is at least as fast as the rate of consumption of electrons by the DME when it is metabolising a candidate drug.

Suitable polymer gels include any large polymer system with delocalised electrons. Preferred polymer gels include carbohydrate gels, such as polysaccharide gels, polypyridine gels, sexithiophene containing gels, and polyaromatic gels.

The polymer gel should have a pore size which is large enough to allow DME molecules to be trapped within the gel matrix. A suitable pore size is 20-50 nm.

Preferably the polymer gel comprises metal binding groups which allow stable binding of the gel to the electrode at the operating voltage of the electrode. Suitable groups include thiols, amides, amines, carboxylic acids, and heterocyclic groups, particularly nitrogen containing heterocyclic groups such as pyridines, purines, pyrimidines, or thiophenes.

Lipid Membranes

Natural CYP enzymes are usually found attached to biological membranes, since they almost exclusively contain a region which acts as an anchor within a phospholipid bilayer. Indeed, the CYPs used in analytical laboratories are generally modified to remove this anchor domain, thus allowing the enzyme to be solubilised. The affinity of CYPs for lipid bilayer membranes provide a means of anchoring them at the surface of an electrode. Suitable membranes may be constructed using long-chain fatty acids, lipids, or similar molecules, deposited on the surface.

Suitable chain lengths are C5-30, preferably C14-22. Branched chains may be advantageous.

Detergents are expected to provide suitable membranes.

Preferably the membrane comprises metal binding groups which allow stable binding of the membrane to the electrode at the operating voltage of the electrode. Suitable metal binding groups include thiols, amides, amines, carboxylic acids, and heterocyclic groups, particularly nitrogen containing heterocyclic groups such as pyridines, purines, pyrimidines, or thiophenes.

A metal binding group may be incorporated along, or at an end of an aliphatic chain.

According to the invention there is also provided use of an electrode of the invention in an electrochemical assay, for example to determine whether a candidate drug, suitably a xenobiotic, is metabolised by the DME immobilised to the electrode.

If the candidate drug acts as a substrate for the DUE, then turnover of the candidate drug by the DME will consume electrons (for example, a Cyp enzyme is expected to consume two electrons per candidate drug molecule if the reaction proceeds via the Cyp catalytic cycle shown in FIG. 4). The rate of consumption of electrons by the Do can be measured using an electrochemical reaction chamber provided that an electrode of the electrochemical reaction chamber supplies electrons to the DME at a rate which is at least as fast as the rate at which they are consumed by the DME (otherwise accurate measurement of the rate of consumption of electrons is not possible since the rate limiting step becomes the transfer of electrons). Ohm's law predicts that if increasing voltage is applied to the electrochemical reaction chamber a constant linear rise in current will occur if there is constant resistance. However, if the candidate drug acts as a substrate for the DME, a deviation from a constant linear rise in current will be seen as electrons are consumed by the reaction. This deviation can be used to calculate the rate of consumption of electrons by the DNM and, therefore, the rate of turnover of the candidate drug by the DME. If this assay is performed for different concentrations of the candidate drug, Vmax and Km can be calculated.

A suitable assay comprises the following steps:

i) providing an electrochemical reaction chamber comprising an electrode of the invention, and a candidate drug;

ii) applying Ghanging voltage to the electrochemical reaction chamber;

iii) measuring current flowing through the electrochemical reaction chamber; and

iv) determining from the measured current whether the candidate drug is metabolised by the DME.

There is also provided according to the invention an electrochemical reaction chamber for carrying out an assay of the invention which comprises an electrode and an electrode of the invention.

There is also provided according to the invention a device comprising a plurality of electrochemical reaction chambers, each electrochemical reaction chamber comprising an electrode and an electrode of the invention, wherein the electrode of the invention for each electrochemical reaction chamber comprises a different DME.

Preferably the electrode and the electrode of the invention are made of the same material such as graphite or metal, preferably gold The electrode may be an electrode of the invention.

The, or each electrochemical reaction chamber is preferably a micro-electrochemical reaction chamber.

It has also been appreciated that efficient transfer of electrons from the electrode(s) to the catalytic site within the DME may be achieved in a system involving a solubilised DME.

According to a second aspect of the invention there is provided an electrode having a surface modified by the covalent or non-covalent addition of chemical groups to allow efficient transfer of electrons from the electrode to a catalytic site within a solubilised DME.

Preferably the chemical groups comprise a delocalised electron system.

The chemical groups preferably comprise a functional group which forms a strong bond to the surface of the electrode, and a functional group for interacting with the solubilised DME.

The electrode binding group should allow formation of a stable bond with the electrode at the operating voltage of the electrode. Preferred metal binding groups for binding to metal electrodes include amides, amines, carboxylic acids, and heterocyclic groups such as thiophenes, or nitrogen containing heterocyclic groups such as pyridines, purines, or pyrimidines.

As with the first aspect of the invention the chemical groups may include sulphur-containing groups such as thiols which have particularly strong affinity for metallic gold. Such groups are preferred where the DME is an FMO, or an oxidative DME other than a CYP.

In a preferred embodiment the electrode is a gold electrode and the chemical groups are organothiloate compounds having an SH group which forms a strong bond to the surface of the electrode, and suitable functional groups for interacting with the solubilised DME.

Many classes of organic molecule provide suitable chemical groups. These include (but are not limited to) metallocenes, flavins, quinones, and NADH.

Preferred chemical groups comprise metallocenes, in particular ferrocenes, as described for the first aspect of the invention. Cobalt metallocenes and vanadium metallocenes are also preferred.

It will be appreciated that the chemical groups must not act as a substrate or an inhibitor of the DME, otherwise accurate measurement of the rate of turnover of a candidate drug by the DME is not possible.

The chemical groups used in electrodes of the invention must have a suitable redox potential for driving the enzyme-catalysed reactions. The importance of the redox potential of the chemical groups and the operating voltage of the electrode of the electrochemical reaction chamber which supplies electrons to the chemical groups is explained below. Preferably the working voltage of the electrode which supplies electrons in the electrochemical reaction chamber is more electronegative than the redox potential of the DME, and the redox potential of the chemical groups is less electronegative than the working voltage of the electrode, but more electronegative than the redox potential of the DME.

An oxidation-reduction (redox) reaction is one where one species loses electrons and another gains them. When a species gains electrons, it is being reduced. When a species loses electrons, it is being oxidized. In all redox reactions, reduction and oxidation occur together: one cannot happen without the other. The electrons flow from one species to the other: there is no net charge gain or loss.

The electrical force produced by an electrochemical cell is measured by the cell voltage, E. Cell voltage depends on the redox reactions occurring in the cell and the concentration of the reactants, but not on the number of electrons passing through the cell.

Since we can split a redox reaction into two parts, we can also define standard voltages for both the oxidation and reduction parts of the reaction, Eₒₓ⁰ and E_{red}⁰. We may arbitrarily pick the hydrogen reduction half reaction
2H⁺_{(aq)}+2e⁻→H_{2(g)}
to have E_{red}⁰=0, and measure all other half reaction voltages in relationship to it. Redox potentials are always given relative to such a reference reaction. In addition to the hydrogen ‘electrode’ shown above, a silver/silver chloride reference is also commonly used, and there are large tables published with the values of standard reduction voltages for half reactions with reference to standard electrode systems. The oxidation half reactions are simply the reaction run in reverse, and the half cell oxidation voltage is the negative of the reduction voltage. Note that the reference electrode in this sense is used to define a ‘baseline’ redox potential to enable redox differences to be quantified. Compounds whose redox potentials differ by, for example, 100 mV will show this same difference no matter what material is used for the reference electrode in the experimental electrochemical cell.

The standard voltage of a cell, E⁰, is the sum of the standard voltages of the oxidation and reduction half reactions. E⁰ is measured when all reactants are at 25° C. and at 1M concentration or 1 atm pressure. The use of the ‘0’ superscript indicates that the values are measured under standard conditions. The addition of an apostrophe, E⁰, indicates that the values are measured under conditions standard for the system being studied. For biological systems, this would be at the relevant physiological conditions of pH, ionic concentration and temperature. To determine if a redox reaction is spontaneous, one should compute the voltage of the reaction. If the voltage is positive, the reaction is spontaneous, and if the voltage is negative, the reaction is not spontaneous.

For the general reaction
aA+bB⇄cC+dD
the equilibrium constant expression has the form
K=[C]^{c}[D]^{d}/[A]^{a}[B]^{b}
where K is the equilibrium constant for the reaction and [X] indicates the concentration of species X. The reaction quotient, Q, is expressed as
Q=[C]^{c} [D]^{d}/[A]^{a}[B]^{b}

The reaction quotient expression of a reaction has the same equation as the equilibrium constant expression for that reaction, however the reaction quotient is computed using the current concentrations, not the equilibrium ones, as indicated by the use of bold type. At equilibrium, Q=K. One use of Q is to determine which way a reaction will go, by computing Q using the current pressures or concentrations and comparing it to K for the reaction. If Q<K, then the reaction will move to the right, and if Q>Kᵢ then the reaction will move to the left.

Since the cell voltage E⁰ determines if the reaction in a cell is spontaneous or not, it clearly must be related to ΔG, the change in the Gibbs free energy. The relationship is
ΔG=−nFE
where n is the number of electrons that are exchanged during the balanced redox reaction and F is the Faraday constant, 9.648×10⁴ C/mol. At standard concentrations at 25° C., this equation can be written as
ΔG⁰=−nFE⁰

Redox reactions like all others can reach an equilibrium state. Since we have a relationship between E⁰ and ΔG⁰ as well as one between ΔG⁰ and K, we can derive a relationship between the cell voltage and the equilibrium constant. Since we have
ΔG⁰=−nFE⁰
and
ΔG⁰=−RT.ln(K)
we can combine the two equations into one:
E⁰=(RT/nF).ln(K)

Under standard conditions, the term RT/F has the value of 0.0257 V, so we can simplify the above equation
E⁰=(0.0257/n).ln(K)

With the above equations, we can derive the value of the cell voltage from the equilibrium constant and vice versa.

We can combine the relationships between ΔG and E at non-equilibrium conditions to get a relationship between the two in much the same way that we can relate K and E at equilibrium. We have the relations
ΔG=ΔG⁰+RT.ln(Q)
ΔG=−nFE
ΔG⁰=−nFE

Combining the three relations gives the Nernst equation
E=E⁰−(RT/nF).ln(Q)

This equation allows us to compute the cell voltage at any concentration of reactants and products and at any temperature. We can simplify the equation slightly by combining constants as before
E=E⁰ −(0.0257/n).ln(Q)

In this invention, the chemical groups accept electrons from the electrode, and are therefore being reduced. The degree to which this occurs can be calculated using the above equations, and it should be clear that the difference between the electrode voltage and the redox potential determines the relative proportions of oxidised and reduced chemical groups in the electrochemical reaction chamber. Thus, the redox potential of the chemical groups and the operating voltage of the electrode are of critical importance in driving the chemical reaction in the direction required. In a similar way, the chemical groups subsequently pass electrons to the DME molecules and are therefore being oxidised. Again, the difference between the redox potentials of the two molecules are crucial in determining the direction, and degree to which, the chemical reaction occurs.

A typical Cyp has a redox potential of −405 mV (vs. an Ag/AgCl reference electrode under standard conditions (E⁰), so this value may be used to determine the preferred redox potentials for suitable chemical groups. According to the standard Cyp catalytic mechanism, the redox potential is lowered by a further 100 mV or so upon substrate binding. Each DME will have a characteristic redox potential, but it is likely that the preferred chemical groups will have potentials falling within the range ±750 mV vs. an Ag/AgCl electrode.

The chemical groups participate in two electrochemical reactions:
Electroded⁻+Chemical groups⇄Electrode+Chemical groups⁻Chemical groups⁻+DME⇄Chemical groups+DME⁻

Both of these reactions must move in the left-to-right direction at a rate that is faster than the rate of the reaction catalysed by the DME, summarised as
RH+O₂+2H⁺+2e⁻→H₂O+ROH

Where R represents the drug.

As has been described, the direction of the electrochemical reactions are determined by the changes in Gibbs free energy, which is related to chemical enthalpy and entropy by the following equation
ΔG=ΔH−T.ΔS

Where ΔH is the change in enthalpy, ΔS is the change in entropy, and T is the reaction temperature.

ΔE is primarily determined by interactions such as chemical (covalent) bonding, electrostatic interactions, hydrogen bonding and van der Waals interactions, not just between the two interacting molecules, but also between each interacting molecule and the solvent. Functional groups which would have a large impact on this component would therefore be those that produce strong interactions of the types listed previously. These include (but are not limited to) amines, amides, carboxylic acids, aromatic systems, heterocycles, enols, ethers, ketones, aldehydes, thiols, thioethers, plus halo-, nitro-, phospho- and sulphate groups.

ΔS is primarily determined by the degrees of freedom in the system, such as the total number of axes along which each molecule may move or rotate, the number of rotatable bonds, the degree of branching in chain-like groups, and the total number of atoms in the system. Again, this component needs to be considered not just between the two interacting molecules, but also between each interacting molecule and the solvent. Functional groups which would have a large impact on this component would therefore be those that contribute to the features listed previously. As before, these include (but are not limited to) amines, amides, carboxylic acids, aromatic systems, heterocycles, enols, ethers, ketones, aldehydes, thiols, thioethers, plus halo-, nitro-, phospho- and sulphate groups.

Many of the interactions described above contribute to the ‘hydrophobic interaction’ component of ΔG, which may be specifically influenced by functional groups such as aromatic systems, hydrogen-bond acceptors and/or donors, and charged groups.

It will be appreciated that the chemical groups should be capable of transferring electrons from the electrode to the DME at a rate which is at least as fast, preferably at least two times as fast, as the rate of consumption of electrons by the DME when a candidate drug is metabolised by the DME. If metabolism of the candidate drug is limited by the transfer of electrons, accurate measurement of the rate of turnover of the candidate drug by the DME is not possible since electron transfer to the DME then becomes the rate-limiting step.

Typically, a DME molecule will turnover approximately 10-100 substrate molecules per second. According to the Cyp catalytic mechanism two electrons are consumed for each molecule of substrate that is turned over. Thus, the chemical groups should be capable of transferring electrons from the electrode to the DME at a rate of at least 20 electrons per second, more preferably at least 40 electrons per second, most preferably at least 200 electrons per second.

Electrodes of the second aspect of the invention may be used in an electrochemical assay, for example to determine whether a candidate drug, suitably a xenobiotic, is metabolised by the DME immobilised to the electrode.

A suitable assay comprises:

i) providing an electrochemical reaction chamber comprising an electrode of the second aspect of the invention, a DNM and a candidate drug in solution;

ii) applying changing voltage to the electrochemical reaction chamber;

iii) measuring current flowing through the electrochemical reaction chamber; and

iv) determining from the measured current whether the candidate drug is metabolised by the DME.

Two of the many possible experimental approaches which are suitable for use in carrying out assays of the first or second aspects of the invention are now described with reference to the accompanying drawings in which:

FIG. 1 shows causes of attrition in drug development (data shown for 198 potential drug candidates which failed during the development stage, taken from Kennedy, T. (1997) Drug Discovery Today 2, pp. 436-444);

FIG. 2 shows the 3D molecular structure of CYP2C9;

FIG. 3 shows the percentage of drugs metabolised by the different CYP families (data shown for 368 drugs which are metabolised by known CYP enzymes, taken from Parkinson, A (1996) Toxicology, McGraw-Hill);

FIG. 4 shows the generally accepted Cyp catalytic cycle;

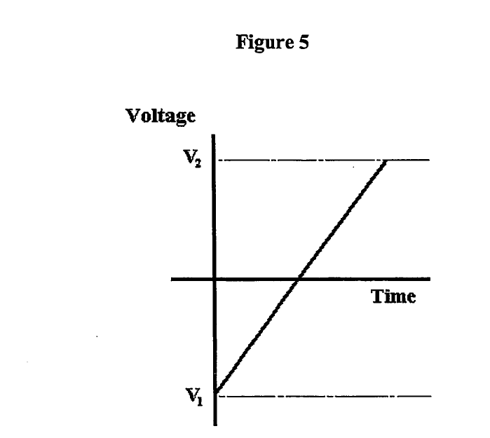

FIG. 5 shows scanning of electrode voltage in linear sweep voltammetry;

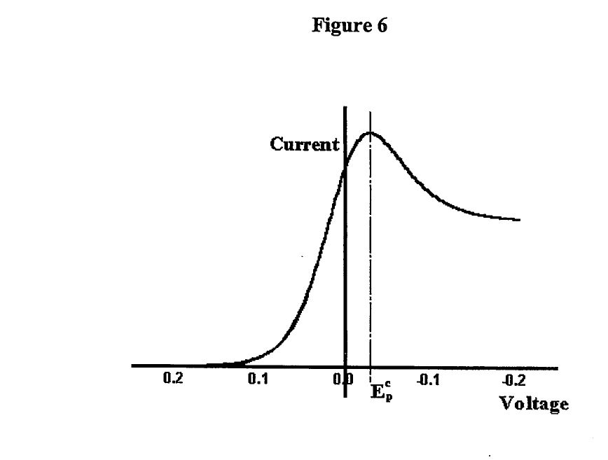

FIG. 6 shows the voltammogram seen for a single voltage scan using an electrolyte solution containing only Fe³⁺ resulting from a voltage sweep;

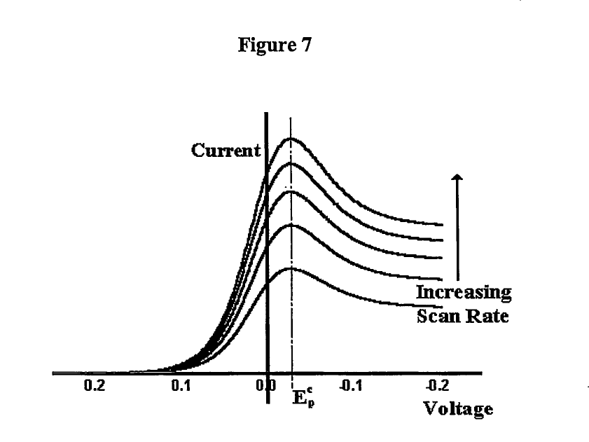

FIG. 7 shows a series of linear sweep voltammograms recorded at different scan rates for an electrolyte solution containing only Fe³⁺;

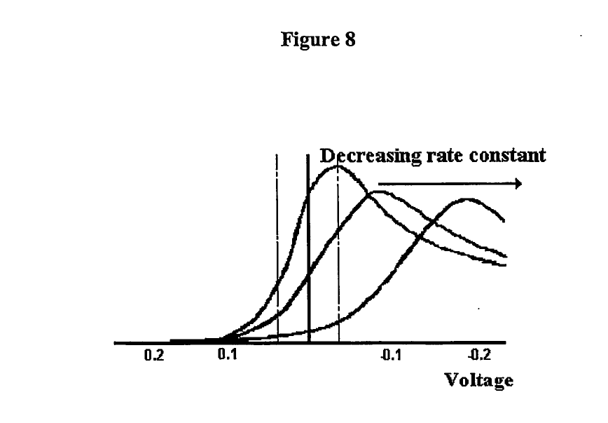

FIG. 8 shows a series of voltammograms recorded at a single voltage sweep rate for different values of the reduction rate constant;

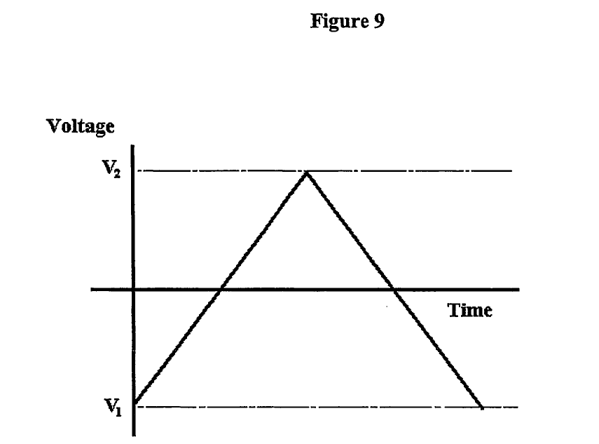

FIG. 9 shows a voltage sweep from V₁ to V₂ at a fixed rate, then back to V₁, as used in cyclic voltammetry,

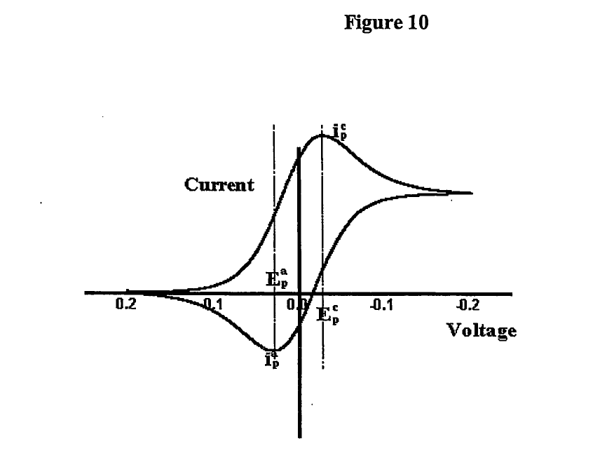

FIG. 10 shows a typical cyclic voltammogram recorded for a reversible single electrode transfer reaction;

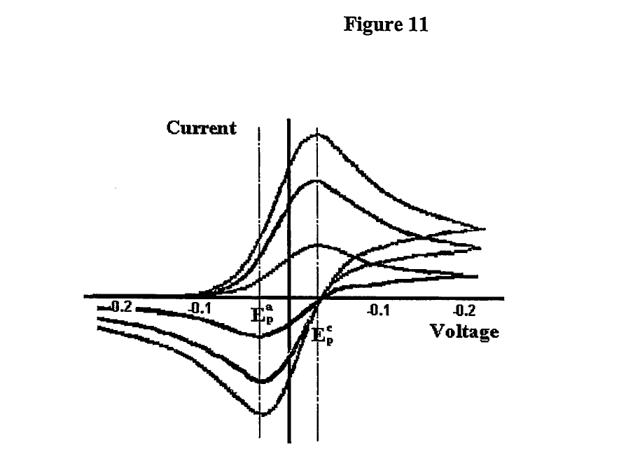

FIG. 11 shows the influence of the voltage scan rate on the current for a reversible electron transfer; and

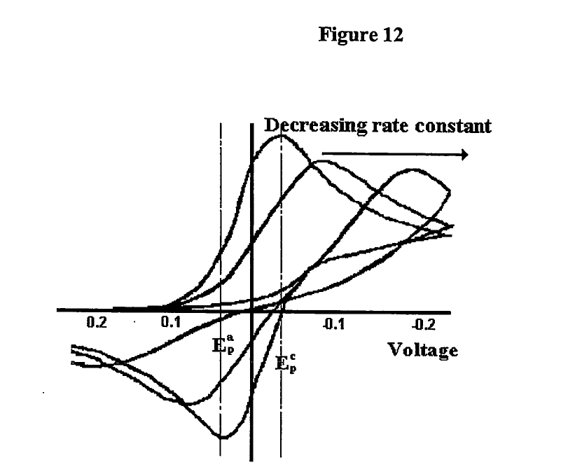

FIG. 12 shows the voltammogram for a quasi-reversible reaction for different values of the reduction and oxidation rate constants.

The reactions are performed in an electrochemical reaction chamber comprising an electrode of the invention. The candidate drug is dissolved in aqueous solution, preferably at a pH, temperature and ionic concentration which closely matches those of standard physiological conditions. Increasing voltage is applied to the electrochemical reaction chamber and the current is measured. The deviation in current from the constant linear rise in current predicted by Ohm's law if resistance is constant is used to calculate the reaction rate for different concentrations of candidate drug. The different reaction rates are then used to calculate the maximum rate (Vmax) of turnover of candidate drug by the DMB, and the concentration of candidate drug (Km) which gives half of Vmax.

The electrochemical reaction chamber may be any suitable size. Bench scale vessels of a few millilitres volume are common, but our preferred reaction chamber would be incorporated into a microfluidics-scale device of a few tens or hundreds of nanoliters. The electrodes may be any suitable material, though our preference would be for gold.

Typical concentrations of the various components are likely to fall in the range 1-100 mM, though more dilute conditions would be preferable.

Linear Sweep Voltammetry (LSV)

In linear sweep voltammetry the electrode voltage is scanned from a lower limit to an upper limit as shown in FIG. 5. The voltage scan rate (v) is calculated from the slope of the line. Clearly by changing the time taken to sweep the range the scan rate is altered.

The characteristics of the linear sweep voltammogram recorded depend on a number of factors including:

The rate of the electron transfer reaction(s)

The chemical reactivity of the electroactive species

The voltage scan rate

In LSV measurements the current response is plotted as a function of voltage rather than time, unlike potential step measurements. For example if we consider the Fe³⁺/Fe²⁺ system

$${Fe}^{3 +} + {ⅇ}^{-} \qquad ⇌ \qquad {Fe}^{2 +}$$

then the voltammogram shown in FIG. 6 would be seen for a single voltage scan using an electrolyte solution containing only Fe³⁺ resulting from a voltage sweep.

The scan begins from the left hand side of the current/voltage plot where no current flows. As the voltage is swept further to the right (to more reductive values) a current begins to flow and eventually reaches a peak before dropping. To rationalise this behaviour we need to consider the influence of voltage on the equilibrium established at the electrode surface. If we consider the electrochemical reduction of Fe³⁺ to Fe²⁺, the rate of electron transfer is fast in comparsion to the voltage sweep rate. Therefore at the electrode surface an equilibrum is established identical to that predicted by thermodynamics. The Nernst equation

$$E = {E}^{e} + \frac{RT}{nF} ln \frac{[ {Fe}^{3 +} ]}{[ {Fe}^{2 +} ]}$$

predicts the relationship between concentration and voltage (potential difference), where E is the applied potential difference and E^{o} is the standard electrode potential. So as the voltage is swept from V₁ to V₂ the equilibrium position shifts from no conversion at V₁ to full conversion at V₂ of the reactant at the electrode surface.

The exact form of the voltammogram can be rationalised by considering the voltage and mass transport effects. As the voltage is initially swept from V¹ the equilibrium at the surface begins to alter and the current begins to flow:

$$\begin{matrix}{Fe}^{3 +} + {e}^{-} & ⟵ & {Fe}^{2 +} \\ {Fe}^{3 +} + {e}^{-} & ⇌ & {Fe}^{2 +} \\ {Fe}^{3 +} + {e}^{-} & ⇌ & {Fe}^{2 +} \\ {Fe}^{3 +} + {e}^{-} & ⇌ & {Fe}^{2 +} \\ {Fe}^{3 +} + {e}^{-} & ⟶ & {Fe}^{2 +}\end{matrix}$$

The current rises as the voltage is swept further from its initial value as the equilibrium position is shifted further to the right hand side, thus converting more reactant. The peak occurs, since at some point the diffusion layer has grown sufficiently above the electrode so that the flux of reactant to the electrode is not fast enough to satisfy that required by the Nernst equation. In this situation the current begins to drop just as it did in the potential step measurements.

The above voltammogram was recorded at a single scan rate. If the scan rate is altered the current response also changes. FIG. 7 shows a series of linear sweep voltammograms recorded at different scan rates for an electrolyte solution containing only Fe³⁺. Each curve has the same form but it is apparent that the total current increases with increasing scan rate. This again can be rationalised by considering the size of the diffusion layer and the time taken to record the scan. Clearly the linear sweep voltammogram will take longer to record as the scan rate is decreased. Therefore the size of the diffusion layer above the electrode surface will be different depending upon the voltage scan rate used. In a slow voltage scan the diffusion layer will grow much further from the electrode in comparison to a fast scan. Consequently the flux to the electrode surface is considerably smaller at slow scan rates than it is at faster rates. As the current is proportional to the flux towards the electrode the magnitude of the current will be lower at slow scan rates and higher at high rates. This highlights an important point when examining LSV (and cyclic voltammograms), although there is no time axis on the graph the voltage scan rate (and therefore the time taken to record the voltammogram) do strongly effect the behaviour seen. A final point to note from FIG. 7 is the position of the current maximum, it is clear that the peak occurs at the same voltage and this is a characteristic of electrode reactions which have rapid electron transfer kinetics. These rapid processes are often referred to as reversible electron transfer reactions.

This leaves the question as to what would happen if the electron transfer processes were ‘slow’ (relative to the voltage scan rate). For these cases the reactions are referred to as quasi-reversible or irreversible electron transfer reactions. FIG. 8 shows a series of voltammograms recorded at a single voltage sweep rate for different values of the reduction rate constant (k_{red}).

In this situation the voltage applied will not result in the generation of the concentrations at the electrode surface predicted by the Nernst equation. This happens because the kinetics of the reaction are ‘slow’ and thus the equilibria are not established rapidly (in comparison to the voltage scan rate). In this situation the overall form of the voltammogram recorded is similar to that shown in FIG. 8, but unlike the reversible reaction now the position of the current maximum shifts depending upon the reduction rate constant (and also the voltage scan rate). This occurs because the current takes more time to respond to the applied voltage than the reversible case.

Cyclic Voltammetry

Cyclic voltammetry (CV) is very similar to LSV. In this case the voltage is swept between two values (see FIG. 9) at a fixed rate, however when the voltage reaches V₂ the scan is reversed and the voltage is swept back to V₁

A typical cyclic voltammogram recorded for a reversible single electrode transfer reaction is shown in FIG. 10. Again the solution contains only a single electrochemical reactant. The forward sweep produces an identical response to that seen for the LSV experiment. When the scan is reversed we simply move back through the equilibrium positions gradually converting electrolysis product (Fe²⁺) back to reactant (Fe³⁺). The current flow is now from the solution species back to the electrode and so occurs in the opposite sense to the forward step but otherwise the behaviour can be explained in an identical manner. For a reversible electrochemical reaction the CV recorded has certain well defined characteristics:

I) The voltage separation between the current peaks is

$$Δ E = {E}_{p}^{a} - {E}_{p}^{c} = \frac{59}{n} mV$$

II) The positions of peak voltage do not alter as a function of voltage scan rate

III) The ratio of the peak currents is equal to one

$$\vert \frac{{\dot{1}}_{p}^{a}}{{\dot{1}}_{p}^{c}} \vert = 1$$

IV) The peak currents are proportional to the square root of the scan rate iₚ^{a} and iₚ^{c}∝\sqrt{v}

The influence of the voltage scan rate on the current for a reversible electron transfer can be seen in FIG. 11. As with LSV the influence of scan rate is explained for a reversible electron transfer reaction in terms of the diffusion layer thickness.

The CV for cases where the electron transfer is not reversible show considerably different behaviour from their reversible counterparts. FIG. 12 shows the voltammogram for a quasi-reversible reaction for different values of the reduction and oxidation rate constants. The first curve shows the case where both the oxidation and reduction rate constants are still fast, however, as the rate constants are lowered the curves shift to more reductive potentials. Again this may be rationalised in terms of the equilibrium at the surface is no longer establishing so rapidly. In these cases the peak separation is no longer fixed but varies as a function of the scan rate. Similarly the peak current no longer varies as a function of the square root of the scan rate. By analysing the variation of peak position as a function of scan rate it is possible to gain an estimate for the electron transfer rate constants.

Application Areas

Ideally, a drug development team would like to have a detailed picture of the pathway and kinetics of a compound's metabolism in humans, including possible side effects such as CYP induction/inhibition and the generation of toxic metabolites, before beginning clinical trials. Gathering as much of this data as possible usually involves a combination of increasingly targeted assay systems. Whole animals are often used for initial toxicological assessment and the outcome of these experiments can prevent a compound from entering the next phase even before any metabolism work is done. Such studies are currently examined using cultured liver cells, live animals or liver slices in combination with a variety or analytical methods to determine the overall metabolic profile. Most recently, such assays are performed using microsomes, synthetic cells comprising isolated enzymes held in an artificial membrane.

Even with the application of increasingly sophisticated analytical methods, there are obvious difficulties in using animals, cells, or cell fractions to obtain information on the specific biochemical events that comprise a compound's metabolism. Advances in the molecular genetics and biochemistry of the DMEs, and the need for greater efficiency in the drug discovery process are driving the development of new in vitro methods based on isolated DMEs. These methods have been used for screening thousands of compounds, and are amenable to integration into the early phases of the drug discovery process. Some of the ways in which recombinant CYPs have been used for in vitro metabolism studies and the rationale for these are described in the following sections. The same general approaches can be applied to other DMEs such as the FMOs, but in most cases the methods are not nearly as well developed as they are for the CYPs.

Isozyme Identification

An identification of the major enzyme(s) involved in your specific drug's metabolism is perhaps the most important component of early studies. Once this is known, pharmacokinetic [PK] studies (see below) are done to obtain Kₘ (an approximate measure of the affinity of the enzyme for the substrate) and Vₘₐₓ (how fast the enzyme can process substrate molecules). Together, these parameters are used to estimate in vivo clearance rates, a key determinant of therapeutic efficacy. Knowledge of the metabolism rate by a specific enzyme may alert the drug discovery team to potential pharmacogenetic problems or drug-drug interactions.

Genetic differences in CYP levels are a major cause of individual variability in response to therapeutics. For example, roughly 8% of the Caucasian population are poor metabolisers of 2D6 substrates and can experience serious side effects when administered normal doses of drugs that are metabolised primarily by this isozyme. Furthermore, some drug-drug interactions can cause serious side effects or even fatal conditions such as drug-induced arrhythmia. The identification of which enzyme is primarily responsible for the metabolism of a drug aids in the design of effective clinical studies used for assessing possible drug interactions. A panel of CYP and FMO enzymes used as biosensors would enable the degree of processing of a new drug by each isozyme to be accurately quantified. This could be achieved using a device according to the invention.

Determination of Kinetic Parameters

Undesirable PK characteristics are frequently a factor in the failure of compounds in preclinical studies. The goal of in vitro studies is to determine the key PK parameters (Kₘ and Vₘₐₓ) for a compound with each CYP isozyme in order to obtain an estimate of the overall in vivo clearance rate. The problems with attempting to obtain accurate kinetic data from crude enzyme preparations such as microsomes include metabolism of the substrate by more than one isozyme, further modification of products (e.g. conjugation), consumption of NADPH by contaminating redox enzymes, and binding of substrates or products to cell proteins or other macromolecules. From an enzymologist's point-of-view, the only way to obtain accurate kinetic data is with isolated enzyme systems. Isolated CYP enzymes used as biosensors would provide this capability.

High-Throughput Screening

A large number of pharmacologically active compounds synthesized in the discovery phase of pharmaceutical R&D are rejected because they interact with the metabolism of existing therapeutic drugs or because they have poor bioavailability caused by rapid metabolism. In many cases, this is because the compounds are either substrates or inhibitors of one or more CYP isozymes. CYPs and other DMEs are generally assayed by isolation and quantification of the metabolites produced from the parent compound. In most cases, this involves chromatographic techniques (usually HPLC) and in some cases phase separations. There are two major drawbacks to these assay methods. First, the need to isolate the reaction products makes the methods too cumbersome and time consuming for use in any type of high-volume assay and precludes the collection of continuous kinetic data. Second, measurement of metabolites requires use of different assay methods for every substrate, raising an obvious technical barrier to screening diverse compounds for metabolism. A universal assay method would be ideal in that it would allow direct quantification of metabolism rates for any substrate, allowing the determination of the key pharmacokinetic parameter (calculated as Vₘₐₓ/Kₘ) for diverse compounds in a high-throughput screening [HTS] format. The intuitive approach for achieving this is to monitor NADPH consumption, which theoretically should be stoichiometric with substrate turnover. However, this has not proven practical because the coupling between NADPH consumption and substrate turnover is variable between different substrates and is frequently as low as 20-30%. Measurement of oxygen consumption suffers from the same drawback; a significant percentage of the total oxygen consumed is diverted into reactive oxygen intermediates rather than metabolite and water.

For these reasons, the main approach that is currently used for screening is competitive inhibition assays, in which inhibition of a probe substrate turnover by the test compound is used to identify potential substrates and inhibitors. The hits from these competitive inhibition screens must be further evaluated to determine whether they are inhibitors or substrates for the indicated isozyme. A number of approaches have been developed for high-throughput screening of CYP inhibition. These techniques include rapid phase separation methods for isolating radiolabeled CYP 2D6 metabolites, development of robotically controlled, multi-column BPLC separation systems to assay testosterone metabolism by CYP 3A4, the use of novel chromogenic reagents for quantitation of formaldehyde formation during CYP-dependent demethylation reactions, and rapid LC/MS approaches for metabolite analysis. However, all of these approaches include relatively cumbersome post-reaction separation steps that limit their usefulness in an HTS format. A lab-on-a-chip style biodetector able to follow CYP mediated reactions at the pharmacokinetic level would not require these separation steps, and so would offer substantial benefits over the current HTS technologies.

1-32. (canceled)

33. A metal electrode comprising a surface at which an oxidative drug-metabolizing enzyme (DME) is immobilized to allow efficient transfer of electrons from the electrode to a catalytic site within the DME.

34. An electrode according to claim 33, wherein the DME is immobilized to the surface of the electrode by means of a linker.

35. An electrode according to either claim 33 or 34, wherein the DME is covalently immobilized to the surface of the electrode.

36. An electrode according to either claim 33 or 34, wherein the DME is non-covalently immobilized to the surface of the electrode.

37. An electrode according to claim 33, wherein the surface of the electrode is modified by covalent or non covalent addition of chemical groups.

38. An electrode according to claim 37, wherein the electrode is a gold electrode and the chemical groups are organothiolate compounds.

39. An electrode according to either claim 33 or 34, wherein the electrode surface is coated with a mechanically and chemically stable polymer gel having high ionic conductivity, and the DME is trapped within the polymer gel.

40. An electrode according to claim 39, wherein the polymer gel comprises polymers having a high proportion of carboxylic acid groups and the DME has positively-charged surface residues.

41. An electrode according to claim 39, wherein the polymer gel comprises polymers having a high proportion of amine groups and the DME has negative charges at the surface.

42. An electrode according to claim 39, wherein the polymer gel comprises polymers having a high proportion of aliphatic groups and the DME has a hydrophobic surface.

43. An electrode according to either claim 33 or 34, wherein the DME is a cytochrome P450 (CYP) which is by means of a lipid membrane deposited on the surface of the electrode.

44. An electrode according to claim 43, wherein the lipid membrane comprises long-chain fatty acids or lipids.

45. An electrode according to claim 34, wherein the linker comprises a delocalized electron system.

46. An electrode according to claim 34 wherein the linker comprises a functional group that is selected from the group consisting of a hydroxyl group, an amide, an amine, a carboxylic acid group, an aromatic group, a cyclic group, a heterocyclic group, a thiophene, a nitrogen-containing heterocyclic group, a pyridine, a purine, a pyrimidine, an enol, an ether, a ketone, an aldehyde, a thiol, a thioether, a halo-, a nitro-, a phospho- and a sulphate group.

47. An electrode according to claim 34 wherein the linker comprises a metallocene, a flavin, a quinone, or NADH.

48. An electrode according to claim 47 wherein the linker comprises a metallocene that comprises a ferrocene.

49. An electrode according to claim 48 wherein the ferrocene is a compound of the following formula: 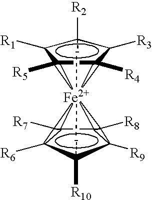
wherein:
R1 is a functional group selected from the group consisting of a thiol, a thioether, an amide, an amine, a carboxylic acid, a heterocyclic group, a thiophene, a nitrogen containing heterocyclic group, a pyridine, a purine and a pyrimidine; and
R₂₋₁₀ are each independently a functional group selected from the group consisting of a hydroxyl group, an amide, an amine, a carboxylic acid group, an aromatic group, a cyclic group, a heterocyclic group, a thiophene, a nitrogen-containing heterocyclic group, a pyridine, a purine, a pyrimidine, an enol, an ether, a ketone, an aldehyde, a thiol, a thioether, a halo-, nitro-, phospho-, and a sulphate group.

50. A metal electrode having a surface modified by covalent or non covalent addition of a chemical group to allow transfer of electrons from the electrode to a catalytic site within a solubilized DME at a rate that is at least as fast as a rate of consumption of electrons by the DME when metabolizing a candidate drug.

51. An electrode according to claim 50 wherein the electrode is a gold electrode and the chemical group comprises an organothiolate compound having (i) an SH group which forms a bond to the surface of the electrode, and (ii) a functional group for interacting with the solubilized DME.

52. An electrode according to claim 51 wherein the chemical group comprises a delocalized electron system.

53. An electrode according to either claim 50 or 52, wherein the chemical group comprises a functional group selected from the group consisting of a hydroxyl group, an amide, an amine, a carboxylic acid group, an aromatic group, a cyclic group, a heterocyclic group, a thiophene, a nitrogen-containing heterocyclic group, a pyridine, a purine, a pyrimidine, an enol, an ether, a ketone, an aldehyde, a thiol, a thioether, a halo-, nitro-, phospho-, and a sulphate group.

54. An electrode according to claim 50 wherein the chemical group comprises a metallocene, a flavin, a quinone, or NADH.

55. An electrode according to claim 54 wherein the chemical group comprises a metallocene that comprises a ferrocene.

56. An electrode according to claim 55, wherein the ferrocene is a compound of the following formula: 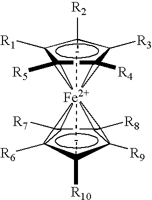
wherein:
R1 is a functional group selected from the group consisting of a thiol, a thioether, an amide, an amine, a carboxylic acid, a heterocyclic group, a thiophene, a nitrogen containing heterocyclic group, a pyridine, a purine, and a pyrimidine; and
R₂₋₁₀ are each independently a functional group selected from the group consisting of a hydroxyl group, an amide, an amine, a carboxylic acid group, an aromatic group, a cyclic group, a heterocyclic group, a thiophene, a nitrogen-containing heterocyclic group, a pyridine, a purine, a pyrimidine, an enol, an ether, a ketone, an aldehyde, a thiol, a thioether, a halo-, nitro-, phospho-, and a sulphate group.

57. An electrochemical reaction chamber comprising a first electrode according to the metal electrode of claim 33; and a second electrode.

58. A device comprising a plurality of electrochemical reaction chambers according to claim 57, wherein the first electrode of each electrochemical reaction chamber comprises a different DME.

59. An electrochemical reaction chamber comprising a first electrode according to the metal electrode of claim 50; a second electrode; and a DME.

60. A device comprising a plurality of electrochemical reaction chambers according to claim 59, wherein the first electrode of each electrochemical reaction chamber comprises a different DME.

61. A method of determining metabolism of a drug by a drug-metabolizing enzyme, comprising:
providing (i) a candidate drug and (ii) a metal electrode that comprises a surface at which an oxidative drug-metabolizing enzyme (DME) is immobilized to allow efficient transfer of electrons from the electrode to a catalytic site within the DME, under conditions that allow transfer of electrons from the electrode to a catalytic site within the DME;
applying changing voltage to the electrode to supply the DME with electrons; and
measuring a rate of consumption of the electrons by the DME, and therefrom determining metabolism of the candidate drug by the DME.

62. A method of determining metabolism of a drug by a drug-metabolizing enzyme, comprising:
providing a candidate drug in solution in an electrochemical reaction chamber, wherein the chamber comprises a metal electrode that comprises a surface at which an oxidative drug-metabolizing enzyme (DME) is immobilized to allow efficient transfer of electrons from the electrode to a catalytic site within the DME, under conditions that allow transfer of electrons from the electrode to a catalytic site within the DME;
applying changing voltage to the electrochemical reaction chamber; and
measuring current flowing through the electrochemical reaction chamber, and therefrom determining metabolism of the candidate drug by the DME.

63. A method of determining metabolism of a drug by a drug-metabolizing enzyme, comprising:
providing (i) a candidate drug and (ii) metal electrode having a surface modified by covalent or non covalent addition of chemical groups to allow transfer of electrons from the electrode to a catalytic site within a solubilized DME at a rate that is at least as fast as a rate of consumption of electrons by the DME when metabolizing a candidate drug;
applying changing voltage to the electrode to supply the DME with electrons; and
measuring a rate of consumption of the electrons by the DME, and therefrom determining metabolism of the candidate drug by the DME.

64. A method of determining metabolism of a drug by a drug-metabolizing enzyme, comprising:
providing a candidate drug in solution in an electrochemical reaction chamber, wherein the chamber comprises a metal electrode having a surface modified by covalent or non covalent addition of chemical groups to allow transfer of electrons from the electrode to a catalytic site within a solubilized DME at a rate that is at least as fast as a rate of consumption of electrons by the DME when metabolizing a candidate drug;
applying changing voltage to the electrochemical reaction chamber; and
measuring current flowing through the electrochemical reaction chamber, and therefrom determining metabolism of the candidate drug by the DME.
